(19) United States
(12) Patent Application Publication
(10) Pub. No.: US 2011/0268683 A1
(43) Pub. Date: Nov. 3, 2011

(54) STRUCTURED SUSPENDING SYSTEMS

(75) Inventors: John Hawkins, South Staffordshire (GB); Pascal J. Herve, West Windsor, NJ (US); Ryan Murphy, Philadelphia, PA (US); Larry Hough, Philadelphia, PA (US)

(73) Assignee: Rhodia, Inc., Cranbury, NJ (US)

(21) Appl. No.: 13/018,169

(22) Filed: Jan. 31, 2011

Related U.S. Application Data
(60) Provisional application No. 61/299,721, filed on Jan. 29, 2010.

(51) Int. Cl. A61K 8/92; A61K 8/46; A61K 33/04; A61K 8/04; A61P 17/00; A61Q 5/12; A61Q 5/02; A61Q 19/10; A61Q 19/00; A61K 8/44; A61K 8/72

(52) U.S. Cl. 424/70.11; 510/123; 510/125; 510/127; 510/119; 510/159; 424/702; 424/70.19; 514/784; 514/770; 514/788; 514/772

(57) ABSTRACT

A pourable ringing gel surfactant composition comprising a mixture of at least one anionic surfactant having a C₈-C₁₈ carbon chain and at least one zwitterionic or amphoteric surfactant having a C₈-C₁₈ carbon chain, wherein the ringing gel is capable of suspending solid or liquid particles without sedimentation.

### CROSS-REFERENCE TO RELATED APPLICATIONS

This application claims priority to U.S. Provisional Application No. 61/299,721, filed Jan. 29, 2010, which is incorporated herein by reference in its entirety.

### FIELD OF THE INVENTION

The invention relates to structured aqueous surfactants having suspending properties. In particular, it provides high foaming suspending systems, suitable for personal care applications, such as shampoos and body-washes.

### BACKGROUND

Formulating suspensions of water insoluble, or sparingly soluble solids and/or liquids in aqueous shampoos presents a long-standing problem. Formulators need to be able to suspend a variety of such ingredients. For example oils, anti-dandruff agents, such as selenium sulphide, hair conditioners including cationic polymers, and opacifiers such as mica are widely used. There is therefore a need to disperse them in aqueous shampoos. The latter, desirably, also comprise anionic and/or amphoteric surfactants, such as alkyl sulfates, sultaines and betaines, which are high foaming and mild to the skin.

We have discovered novel structured surfactant systems that are capable of suspending solid particles and oils without sedimentation, using high foaming surfactants which give dense stable foams with good wetting and feel.

The term “structured system” as used herein means a Pourable composition comprising water, surfactant, any structurants, which may be required to impart suspending properties to the surfactant, and optionally other dissolved matter, which together form a mesophase, or a dispersion of a mesophase in a continuous aqueous medium, and which has the ability to immobilise non-colloidal, water-insoluble particles, while the system is at rest, thereby forming a stable, Pourable suspension.

The term “Pourable” is used herein to mean approximately that 100 mL of gel in a 200 mL jar, when inverted, will flow substantially to the bottom of the jar in less than 30 minutes.

Attempts to solve the problem of dispersing water insoluble materials in water have generally involved either using gums or other polymeric thickeners to raise the viscosity of the liquid medium, or else forming colloidal dispersions. More recently the use of lamellar structured surfactants has been proposed.

Gums and polymeric thickeners, which increase the viscosity of the liquid medium, retard, but do not prevent sedimentation, and at the same time make the composition harder to pour. They do not provide stable suspensions.

Colloidal dispersions that contain particles of about 1 micron or smaller are prevented from sedimenting by Brownian motion. Such systems are obviously incapable of dispersing relatively coarse particles. They are moreover not fully stable, because the dispersed particles tend to grow in size, due to agglomeration and Ostwald ripening, until they are too large to be maintained in suspension.

Lamellar structured suspending systems depend on the rheological properties of the suspending medium to immobilise the particles, irrespective of size. This requires the suspending medium to exhibit a yield point, which is higher than the sedimenting or creaming force exerted by the suspended particles, but low enough to enable the medium to flow under externally imposed stresses, such as pouring and stirring, like a normal liquid. The structure reforms sufficiently rapidly to prevent sedimentation, once the agitation caused by the external stress has ceased.

Three main types of lamellar structured system have been employed in practice, all involving an L_{α}-phase, in which bilayers of surfactant are arranged with the hydrophobic part of the molecule on the interior and the hydrophilic part on the exterior of the bilayer (or vice versa). The bilayers lie side by side, e.g. in a parallel or concentric configuration, sometimes separated by aqueous layers. L_{α}-phases (also known as G-phases) can usually be identified by their characteristic textures under the polarising microscope and/or by x-ray diffraction, which is often able to detect evidence of lamellar symmetry. Such evidence may comprise first, second and sometimes third order peaks with a d-spacing (2π/Q, where Q is the momentum transfer vector) in a simple integral ratio 1:2:3. Other types of symmetry give non-integral ratios. The d-spacing of the first peak in the series corresponds to the repeat spacing of the bilayer system.

Most surfactants form an L_{α}-phase either at ambient or at some higher temperature when mixed with water in certain specific proportions. However such conventional L_{α}-phases do not usually function as structured suspending systems. Useful quantities of solid render them unpourable and smaller amounts tend to sediment.

The main types of structured system used in practice are based on dispersed lamellar, spherulitic and expanded lamellar phases. Dispersed lamellar phases are two phase systems, in which the surfactant bilayers are arranged as parallel plates to form domains of L_{α}-phase, which are interspersed with an aqueous phase to form an opaque gel-like system. They are described in EP 0 086 614.

Spherulitic phases comprise well-defined spheroidal bodies, usually referred to in the art as spherulites, in which surfactant bilayers are arranged as concentric shells. The spherulites usually have a diameter in the range 0.1 to 15 microns and are dispersed in an aqueous phase in the manner of a classical emulsion, but interacting to form a structured system. Spherulitic systems are described in more detail in EP 0 151 884.

Many structured systems are intermediate between dispersed lamellar and spherulitic, involving both types of structure. Usually systems having a more spherulitic character are preferred because they tend to have lower viscosity. A variant on the spherulitic system comprises prolate or rod shaped bodies sometimes referred to as batonettes. These are normally too viscous to be of practical interest. For example, the M phase is formed by rod-shaped micelles, is anisotropic, generally cloudy, highly viscous, and is non-suspending.

Both of the foregoing systems comprise two phases. Their stability depends on the presence of sufficient dispersed phase to pack the system so that the interaction between the spherulites or other dispersed mesophase domains prevents separation. If the amount of dispersed phase is insufficient, e.g. because there is not enough surfactant or because the surfactant is too soluble in the aqueous phase to form sufficient of a mesophase, the system will undergo separation and cannot be used to suspend solids. Such unstable systems are not “structured” for the purpose of this specification.

A third type of structured system comprises an expanded L_{α}-phase. It differs from the other two types of structured system in being essentially a single phase, and from conventional L_{α}-phase in having a wider d-spacing. Conventional L_{α}-phases, which typically contain 60 to 75% by weight surfactant, have a d-spacing of about 4 to 7 nanometers. Attempts to suspend solids in such phases result in stiff pastes which are either non-pourable, unstable or both. Expanded L_{α}-phases with d-spacing greater than 8, e.g. 10 to 15 nanometers, form when electrolyte is added to aqueous surfactants at concentrations just below those required to form a normal

L_{α}-phase, particularly to surfactants in the H-phase.

The H-phase comprises surfactant molecules arranged to form cylindrical rods of indefinite length. It exhibits hexagonal symmetry and a distinctive texture under the polarising microscope. Typical H-phases have so high a viscosity that they appear to be curdy solids. H-phases near the lower concentration limit (the L₁/H-phase boundary) may be pourable but have a very high viscosity and often a mucus-like appearance. Such systems tend to form expanded L_{α}-phases particularly readily on addition of sufficient electrolyte.

Expanded L_{α}-phases are described in more detail in EP 0 530 708. In the absence of suspended matter they are generally translucent, unlike dispersed lamellar or spherulitic phases, which are normally opaque. They are optically anisotropic and have shear-dependent viscosity. In this they differ from L₁-phases, which are micellar solutions or microemulsions. L₁-phases are clear, optically isotropic and are usually substantially Newtonian. They are unstructured and cannot suspend solids.

Some L₁-phases exhibit small angle x-ray diffraction spectra, which show evidence of hexagonal symmetry and/or exhibit shear dependent viscosity. Such phases usually have concentrations near the L₁/H-phase boundary and may form expanded L_{α}-phases on addition of electrolyte. However in the absence of any such addition of electrolyte they lack the yield point required to provide suspending properties, and are not, therefore, “structured systems” for the purpose of this specification.

Expanded L_{α}-phases of the above type are usually less robust than spherulitic systems. They are liable to become unstable at low temperatures. Moreover they frequently exhibit a relatively low yield stress, which may limit the maximum size of particle that can be stably suspended.

Most lamellar structured surfactants require the presence of a structurant, as well as surfactant and water in order to form structured systems capable of suspending solids. The term “structurant” is used herein to describe any non-surfactant capable, when dissolved in water, of interacting with surfactant to form or enhance (e.g. increase the yield point of,) a structured system. It is typically a surfactant-desolubiliser, e.g. an electrolyte. However, certain relatively hydrophobic surfactants such as isopropylamine alkyl benzene sulphonate can form spherulites in water in the absence of electrolyte. Such surfactants are capable of suspending solids in the absence of any structurant, as described in EP 0 414 549.

It is known from WO 01/05932 that carbohydrates can interact with surfactants to form lamellar suspending structures. Such systems generally exhibit even greater d-spacings than the electrolyte-structured expanded L_{α}-phases, described in EP 0 530 708. The d-spacings of the sugar-structured systems, described in WO 01/05932, are typically greater than 15 nm, and may, for example, be as high as 50 nm. Such systems can be obtained in a clear or translucent form by suitable choice of surfactant and carbohydrate concentration.

Optically clear lamellar systems were also described in EP 1 141 121, which proposed using deflocculating polymers to shrink the lamellar droplets (spherulites) to a diameter too small to interact with visible light, or using sugar to adjust the refractive index of the continuous phase to match that of the spherulites.

A major problem with lamellar suspending systems, from the point of view of the formulator of personal care products, is that they are formed most readily by surfactant systems having a mean HLB in the range 9 to 12, which includes those surfactants which are most effective in domestic products such as laundry detergents and hard surface cleaners, but excludes the higher foaming surfactants, which are most effective in personal care products, such as shampoos. The latter generally have higher HLB values.

Attempts to form stable lamellar suspending systems with high foaming surfactants have entailed the use of high concentrations of surfactant and high levels of structurant, such as electrolyte or sugar, and/or mixing the high HLB surfactants with low HLB surfactants, to lower the mean HLB.

In general the use of high surfactant levels, e.g. greater than about 15-20% by weight, is undesirable on grounds both of cost and the potential for producing adverse effects on skin or hair. High electrolyte levels are similarly undesirable, for their potential effects on skin and hair, and because they inhibit foaming. Sugar generally needs to be present in undesirably high concentrations, e.g. over 20% to be effective. Low HLB surfactants inhibit foaming.

### SUMMARY OF THE INVENTION

I have now discovered a novel, structured suspending system based on a non-lamellar surfactant mesophase, which is most readily formed from relatively high HLB surfactants, in lower concentrations than those required for lamellar phases, and which can tolerate, high levels of electrolyte.

Most anionic and amphoteric surfactants, which have HLBs above 10, and in the absence of structurants, show the same typical progression as the concentration is increased above the critical micellar concentration. They initially form an L₁-phase comprising spherical micelles at relatively low concentrations. As the concentration is increased, the viscosity increases slightly. At higher concentrations a transition normally occurs from spherical to prolate (rod) micelles marked by a more rapid increase in viscosity with concentration. Eventually the L₁/H-phase boundary is reached, typically at about 20-40% by weight surfactant and an immobile, hexagonal H-phase is obtained. Finally, at concentrations above about 30-60%, the H/L_{α}-phase boundary is encountered and a pourable, but viscous, lamellar L_{α}-phase is formed.

The presence of structurants tends to lower the concentration at the phase boundaries and suppress the hexagonal, H-phase, giving a more dilute and more mobile L_{α}-phase.

In addition to the lamellar and hexagonal symmetries, which characterise respectively the L_{α} and H-phases discussed above, there is a third possible symmetry, which some surfactant mesophases have been found to exhibit. A group of phases, usually referred to collectively as “Viscous Isotropic” or VI-phases, are known, some of which exhibit cubic symmetry, and others of which may exhibit, for example, cross-linked or non-cross-linked wormlike micellar structure. Typically, all of these VI-phases are called “ringing gels,” because of a distinctive vibration, due to their highly elastic character, that can be felt when they are struck a sharp blow.

As their name implies, VI-phases are clear, optically isotropic gels, which are usually highly viscous and often viscoelastic, and have therefore largely been ignored by formulators. They have hitherto mainly been encountered with non-ionic surfactant systems, which are too harsh to be ideal for personal care products.

Four different cubic phases distinguishable among the VI phases are called the V₁-, I₁-, I₂- and V₂-phases respectively. The two I-phases are bicontinuous and take the form of rigid gels. The V₁-phase is generally envisaged as comprising a close packed cubic array of spherical surfactant micelles. The V₂-phase is the inverse of the V₁ with water as the dispersed phase, and is generally less rigid than the I-phases.

I have now discovered that certain mixtures of anionic, zwitterionic and/or amphoteric surfactants form a Pourable isotropic gel, with suspending properties, at concentrations below those corresponding to the lamellar phase. The system is typically clear and optically isotropic, like an L₁-phase, but has suspending properties like an L_{α}-phase. At least at concentrations in the upper part of its range, “ringing” properties typical of VI-phases are clearly observable when samples of the novel phase are struck.

Viscoelastic surfactant fluids may form worm-like, rod-like, or cylindrical micelles in solution. The formation of long, cylindrical micelles creates useful rheological properties. The viscoelastic surfactant solution exhibits shear thinning behaviour, and remains stable despite repeated high shear applications. By comparison, the typical polymer thickener will irreversibly degrade when subject to high shear. Without wishing to be bound by any theory, it is thought that the novel system is a cubic phase or a viscoelastic worm-like micellar system.

It has surprisingly and unexpectedly been discovered that surfactants typically used in personal care products may form ringing gels. It has further surprisingly and unexpectedly been discovered that ringing gels may be formulated having advantageous properties such as suspending and flowability properties. It has further unexpectedly been discovered that ringing gel melting points and/or gel strength may be predicted and/or modified based on gel salt content.

According to a first embodiment the invention provides a novel aqueous suspending system, which comprises a mixture of anionic, zwitterionic and/or amphoteric surfactants in the form of a Pourable V₁-phase.

According to a second embodiment, the invention provides a novel aqueous suspending system, which comprises a mixture of anionic, zwitterionic and/or amphoteric surfactants in the form of a Pourable ringing gel.

According to a third embodiment, the invention provides the use of an aqueous suspending system as aforesaid to suspend solid and/or liquid particles.

According to a further embodiment the invention provides a personal care product comprising an aqueous suspending system as aforesaid and suspended solid and/or liquid particles.

In particular I have noted that my novel suspending phase is most readily formed by non-cyclic, e.g. essentially linear, molecules.

I have further noticed that my novel suspending phase is most readily formed by mixtures of longer and shorter chain molecules.

I have further noticed that my novel suspending phase is most preferably formed by a mixture of at least one anionic with at least one amphoteric and/or zwitterionic surfactant.

In some embodiments, a the composition comprises a gel with equimolar amounts of salt and surfactant.

In some embodiments, the amount of salt used to give a gel with a high melting point and/or high strength may be predicted by the molecular weight of the component surfactants.

In some embodiments, the gel has a structure such as is shown in FIG. 4.

In some embodiments, the gel comprises about 20% of total surfactant comprising equimolar amounts of anionic and amphoteric surfactants, and sufficient electrolyte to balance charges on the surfactants.

In some embodiments, the invention relates to a composition comprising a mixture of at least one anionic surfactant and at least one zwitterionic or amphoteric surfactant, wherein:
said at least one anionic surfactant comprises a C₈-C₁₈ carbon chain;
said at least one zwitterionic or amphoteric surfactant comprises a C₈-C₁₈ carbon chain; and
said composition comprises a Pourable ringing gel capable of suspending solid or liquid particles without sedimentation.

In some embodiments, at least one of the carbon chains is at least partially unsaturated.

In some embodiments, at least one of the carbon chains is saturated.

In some embodiments, at least one of the carbon chains is linear.

In some embodiments, at least one of the carbon chains is branched.

In some embodiments, the surfactants of the invention do not comprise carbon chains having more than 18 carbon atoms.

In some embodiments, the surfactants of the invention do not comprise carbon chains having more than 16 carbon atoms.

In some embodiments, the surfactants of the invention do not comprise carbon chains having more than 14 carbon atoms.

In some embodiments, the at least one anionic surfactant comprises a C₁₀-C₁₆ carbon chain.

In some embodiments, the at least one anionic surfactant comprises a C₁₂-C₁₄ carbon chain.

In some embodiments, the at least one anionic surfactant comprises a C₁₂₋₁₄ alkyl sulfate, an oleyl taurate, or a mixture thereof.

In some embodiments, the anionic surfactant comprises a dodecanoate, myristate, stearate, isostearate, oleate, linoleate, linolenate, behenate, erucate, palmitate, coconut or tallow soap; olefin sulphonate; paraffin sulphonate; isethionate; sarcosinate; sulphosuccinate; sulphosuccinamate, or a combination thereof.

In some embodiments, the anionic surfactant is not an ethoxylated surfactant.

In some embodiments, at least one at least one zwitterionic or amphoteric surfactant comprises a C₁₀-C₁₆ carbon chain.

In some embodiments, the at least one zwitterionic or amphoteric surfactant comprises a C₁₂-C₁₄ carbon chain.

In some embodiments, the at least one zwitterionic or amphoteric surfactant comprises an imidazoline betaine.

In some embodiments, the at least one zwitterionic or amphoteric surfactant comprises a compound of formula:

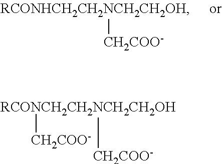

wherein R comprises said C₈-C₁₈ carbon chain.

In some embodiments, the carbon chain comprises a mixture of alkyl and alkenyl groups.

In some embodiments, the zwitterionic surfactant comprises a betaine, sulfobetaine or hydroxy sulfobetaine.

In some embodiments, the zwitterionic surfactant comprises a compound of formula:

R″R′₂ NCH₂XOH
wherein:
X comprises C═O or SO₂,
R′ comprises an aliphatic group having 1 to 4 carbon atoms, and
R″ comprises an aliphatic group having from 8 to 18 carbon atoms.

In some embodiments, R″ is branched.

In some embodiments, R″ comprises a straight chain alkyl or alkenyl group.

In some embodiments, R″ comprises a group of formula:

RCONR′(CH₂)ₙ
wherein:
R comprises said C₈-C₁₈ carbon chain
R′ comprises an aliphatic group having 1 to 4 carbon atoms, and
n is an integer ranging from 2 to 4.

In some embodiments, the zwitterionic surfactant comprises a compound of formula:

R″R′₂NCH₂XOH

wherein:
X comprises C═O or SO₂,
R′ comprises a methyl, carboxymethyl, ethyl, hydroxyethyl, carboxyethyl, propyl, isopropyl, hydroxypropyl, carboxypropyl, butyl, isobutyl or hydroxybutyl group, and
R″ comprises an aliphatic group having from 8 to 18 carbon atoms.

In some embodiments, the mixture further comprises a nonionic surfactant comprising an amine oxide, a polyglyceryl fatty ester, a fatty acid ethoxylate, a fatty acid monoalkanolamide, a fatty acid dialkanolamide, a fatty acid alkanolamide ethoxylate, a propylene glycol monoester, a fatty alcohol propoxylate, an alcohol ethoxylate, an alkyl phenol ethoxylate, a fatty amine alkoxylate, a fatty acid glyceryl ester ethoxylate, a mixed ethylene oxide/propylene oxide block copolymer, an ethylene glycol monoester, an alkyl polyglycoside, an alkyl sugar ester, an ethoxylated sorbitan ester, an alkyl capped polyvinyl alcohol, an alkyl capped polyvinyl pyrrolidone, or a combination thereof.

In some embodiments, the composition comprises at least one electrolyte.

In some embodiments, the electrolyte comprises sodium, potassium and/or ammonium chloride; sodium or potassium carbonate; sodium phosphate; sodium citrate, sugar, or a combination thereof.

In some embodiments, the electrolyte comprises sodium chloride.

In some embodiments, the total amount of electrolyte is less than 10% by weight of the composition.

In some embodiments, the total amount of electrolyte is less than 5% by weight of the composition.

In some embodiments, the gel comprises about 20% of total surfactant comprising equimolar amounts of anionic and amphoteric surfactants, and sufficient electrolyte to balance charges on the surfactants.

In some embodiments, the total amount of surfactant comprises greater than 2% of the composition by weight.

In some embodiments, the total amount of surfactant comprises greater than 4% of the composition by weight.

In some embodiments, the total amount of surfactant comprises greater than 6% of the composition by weight.

In some embodiments, the total amount of surfactant comprises greater than 8% of the composition by weight.

In some embodiments, the total amount of surfactant comprises greater than 15% of the composition by weight.

In some embodiments, the total amount of surfactant ranges from 8% to 30% of the composition by weight.

In some embodiments, the weight ratio of the at least one anionic surfactant to the at least one amphoteric or zwitterionic surfactant ranges from 1:10 to 10:1.

In some embodiments, the weight ratio of the at least one anionic surfactant to the at least one amphoteric or zwitterionic surfactant ranges from 1:5 to 5:1

In some embodiments, the weight ratio of the at least one anionic surfactant to the at least one amphoteric or zwitterionic surfactant ranges from 1:2 to 2:1

In some embodiments, the weight ratio of the at least one anionic surfactant to the at least one amphoteric or zwitterionic surfactant ranges from 1:1.5 to 1.5:1

In some embodiments, the invention relates to a composition that is a component in a personal care product.

In some embodiments, the invention relates to a composition comprising a personal care product.

In some embodiments, the personal care product comprises a shampoo, shaving gel, bath gel, shower gel, cosmetic cream, ointment, or exfoliant.

In some embodiments, the surfactants of the invention form a worm-like micellar structure.

In some embodiments, the surfactants of the invention form a structure comprising a cubic symmetry.

In some embodiments, the composition of the invention is clear and optically isotropic.

In some embodiments, the composition of the invention is shear thinning.

In some embodiments, the composition of the invention comprises suspended solid or liquid particles.

In some embodiments, the solid or liquid particles comprise an oil, an anti-dandruff agent, a hair conditioner, an opacifier, mica, or a metallic flake.

In some embodiments, the hair conditioner comprises a cationic polymer.

In some embodiments, the particles comprise an anti-dandruff agent.

In some embodiments, the anti-dandruff agent comprises selenium sulfide.

In some embodiments, the particles comprise mica.

In some embodiments, the composition of the invention has a viscosity greater than 10 Pa*s, preferably greater than 100 Pa*s, more preferably greater than 1000 Pa*s, at 2% total surfactant concentration when the viscosity is measured at a shear rate of 1×10⁻³ s⁻¹ with a rheometer comprising a cross-hatched 40 mm diameter parallel plate and a gap of 0.8 mm.

In some embodiments:
the at least one anionic surfactant comprises a C₁₀-C₁₆ carbon chain, and/or
the at least one zwitterionic or amphoteric surfactant comprises a C₁₀-C₁₆ carbon chain; and
the composition has a viscosity greater than 10 Pa*s, preferably greater than 100 Pa*s, more preferably greater than 1000 Pa*s, at 2% total surfactant concentration when the viscosity is measured at a shear rate of 1×10⁻³ s⁻¹ with a rheometer comprising a cross-hatched 40 mm diameter parallel plate and a gap of 0.8 mm.

In some embodiments:
the at least one anionic surfactant comprises a C₁₂-C₁₄ carbon chain, and/or
the at least one zwitterionic or amphoteric surfactant comprises a C₁₂-C₁₄ carbon chain; and
the composition has a viscosity greater than 10 Pa*s, preferably greater than 100 Pa*s, more preferably greater than 1000 Pa*s, at 2% total surfactant concentration when the viscosity is measured at a shear rate of 1×10⁻³ s⁻¹ with a rheometer comprising a cross-hatched 40 mm diameter parallel plate and a gap of 0.8 mm.

In some embodiments, the composition of the invention has a pH ranging from 4 to 9.

In some embodiments, the composition of the invention has a pH ranging from 6 to 8.

In some embodiments, the composition comprises a mixture of sodium lauryl sulfate, cocoamidopropylhydroxysultaine, and sodium chloride in an aqueous medium,
wherein said composition comprises a Pourable ringing gel capable of suspending solid or liquid particles, and
further wherein said composition has a viscosity greater than 10 Pa*s, preferably greater than 100 Pa*s, more preferably greater than 1000 Pa*s, at 2% total surfactant concentration when the viscosity is measured at a shear rate of 1×10⁻³ s⁻¹ with a rheometer comprising a cross-hatched 40 mm diameter parallel plate and a gap of 0.8 mm.

In some embodiments, the invention relates to a method of making a surfactant composition comprising:
mixing at least one anionic surfactant and at least one zwitterionic or amphoteric surfactant in an aqueous medium, wherein:
   said at least one anionic surfactant comprises a C₈-C₁₈ carbon chain;
   said at least one zwitterionic or amphoteric surfactant comprises a C₈-C₁₈ carbon chain; and
   said composition comprises a Pourable ringing gel capable of suspending solid or liquid particles without sedimentation.

In some embodiments, the invention relates to a method for suspending solid or liquid particles comprising mixing said particles with a composition comprising a mixture of at least one anionic surfactant and at least one zwitterionic or amphoteric surfactant, wherein:
said at least one anionic surfactant comprises a C₈-C₁₈ carbon chain;
said at least one zwitterionic or amphoteric surfactant comprises a C₈-C₁₈ carbon chain; and
said composition comprises a Pourable ringing gel capable of suspending said particles without sedimentation.

### BRIEF DESCRIPTION OF THE FIGURES

FIG. 1 shows a phase diagram of the w/w ratio of Sulfate:Betaine versus percent actives with 0% NaCl.

FIG. 2 shows a phase diagram of the w/w ratio of Sulfate:Betaine versus percent actives with 3% NaCl.

FIG. 3 is a plot of gel set point versus salt concentration.

FIG. 4 shows a gel structure for some embodiments.

FIG. 5 shows a phase diagram of the w/w ratio of SLE(2)S:Betaine versus percent actives

FIG. 6 is a plot of viscosity versus shear rate for a 5 wt % SLS-Sultaine gel with various SLS weight fractions.

FIG. 7 is a plot of modulii versus angular frequency for a 5 wt % SLS-Sultaine gel with a 0.4 SLS weight fraction.

FIG. 8 is a plot of viscosity versus shear rate for a 10 wt % SLS-Sultaine gel with various SLS weight fractions.

### DETAILED DESCRIPTION OF PREFERRED EMBODIMENTS

In the following discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The surfactant system is typically a high foaming mild surfactant, which may comprise anionic, amphoteric, and/or zwitterionic surfactants. Mixtures of anionic with, zwitterionic and/or amphoteric surfactants are particularly preferred.

In some embodiments, the surfactant systems may have a mean HLB greater than 40.

The surfactant preferably comprises a mixture of an anionic surfactant with an amphoteric surfactant, and/or of a long chain surfactant with a short chain surfactant, for example mixtures of C₁₂₋₁₄ alkyl sulfate with oleyl taurates and/or amphoteric surfactants such cocoamidopropyl betaine or sulphobetaine, or mixtures of taurates with amphoteric surfactant.

The preferred anionic surfactants are C₁₂₋₁₄ alkyl sulfates and taurates. Other anionic surfactants, which may be present include, for example: C₈₋₂₀ e.g. C₁₀₋₂₀ aliphatic soaps, such as dodecanoates, myristates, stearates, isostearates, oleates, linoleates, linolenates, behenates, erucates, palmitates, coconut and tallow soaps; olefin sulphonates; paraffin sulphonates; isethionates; sarcosinates; sulphosuccinates; or sulphosuccinamates.

Preferably, the anionic surfactants comprise a carbon chain having from 8 to 18 carbon atoms, more preferably from 10 to 16 carbon atoms, still more preferably from 12 to 14 carbon atoms.

I have found that my novel suspending system is less readily formed in the presence of ethoxylated surfactants such as ether sulfates and alkaryl or alicyclic surfactants, such as alkylbenzene sulphonate. I do not exclude the presence of such surfactants but prefer that they constitute less than 50% by weight of the anionic surfactant, more preferably less than 20%, still more preferably less than 10%, most preferably that they be essentially absent. It has been discovered that ringing gels containing significant amounts of ethoxylated surfactant can be prepared if a heating stage is included in the preparative method. It is thought that this is because at room temperatures, ethoxylated surfactant chains have water hydrogen bonded to them and this does not allow them to pack into spherical micelles. However, when the water is removed by heating, such as greater than 30° C., then the ethoxylated surfactants will pack into micelles and ringing gels are observed. In this regard, it was observed that the ringing gels comprising ethoxylated surfactant reform during cooling from above 30° C., and once formed, the ethoxylated surfactant chains are substantially no longer available for hydrogen bonding by water molecules.

The cation of the anionic surfactant is typically sodium and/or ammonium. However it may alternatively or additionally comprise potassium, lithium, calcium, magnesium, ammonium, or an alkyl or hydroxyalkyl ammonium having up to 6 aliphatic carbon atoms including ethylammonium, isopropylammonium, monoethanolammonium, diethanolammonium, and triethanolammonium, or a mixture of any of the foregoing.

The surfactant may optionally comprise minor proportions non-ionic surfactants such as amine oxides, polyglyceryl fatty esters, fatty acid ethoxylates, fatty acid monoalkanolamides, fatty acid dialkanolamides, fatty acid alkanolamide ethoxylates, propylene glycol monoesters, fatty alcohol propoxylates, alcohol ethoxylates, alkyl phenol ethoxylates, fatty amine alkoxylates and fatty acid glyceryl ester ethoxylates. Other non-ionic compounds suitable for inclusion in compositions of the present invention include mixed ethylene oxide/propylene oxide block copolymers, ethylene glycol monoesters, alkyl polyglycosides, alkyl sugar esters including alkyl sucrose esters and alkyl oligosaccharide esters, sorbitan esters, ethoxylated sorbitan esters, alkyl capped polyvinyl alcohol and alkyl capped polyvinyl pyrrolidone.

The surfactant preferably comprises an amphoteric and/or zwitterionic surfactant. The former may comprise so-called imidazoline betaines, also called amphoacetates, which were traditionally ascribed the zwitterionic formula:

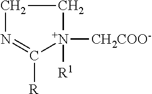

because they are obtained by reacting sodium chloracetate with an imidazoline. It has been shown, however, that they are actually present, at least predominantly, as the corresponding, amphoteric, linear amidoamines:

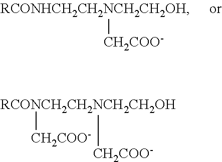

R preferably has at least 8, more preferably at least 10 carbon atoms but less than 25, more preferably less than 22, even more preferably less than 20, most preferably less than 18. Typically R represents a mixture of alkyl and alkenyl groups, obtained, for example, from coconut or palm oil, and having sizes ranging from 8 to 18 carbon atoms, with 12 predominating, or a fraction of such a feedstock, such as lauryl (>90% C₁₂).

The zwitterionic surfactant is preferably a betaine, sulphobetaine or hydroxy sulphobetaine, e.g. one with the formula R″R′₂ NCH₂XOH, where X is CO or SO₂, each R′ is an aliphatic group having 1 to 4 carbon atoms and R″ is an aliphatic group having from 8 to 25 carbon atoms (preferably 8 to 18 carbon atoms), preferably a branched or more preferably, straight chain alkyl or alkenyl group, or more preferably a group of the formula RCONR'(CH₂)ₙ, where R and R′ have the same significance as before, and n is an integer from 2 to 4.

I prefer that R′ is a methyl, carboxymethyl, ethyl, hydroxyethyl, carboxyethyl, propyl, isopropyl, hydroxypropyl, carboxypropyl, butyl, isobutyl or hydroxybutyl group.

Surfactant systems of my invention may show significant increase in viscosity when the total active concentration is raised above about 2%, preferably above about 4% by weight. At active concentrations above about 4%, preferably above about 6%, a yield point is observed and/or sufficient viscosity is exhibited, which may be sufficient to suspend relatively low density materials, such as talc. As the concentration is raised further the viscosity and/or yield point rise to a maximum, with the ability to suspend high density solids, but then decline as the composition becomes turbid and anisotropic.

Preferably the total surfactant is present in a proportion of greater than 8, more preferably greater than 13, most preferably greater than 15% by weight based on the total weight of the formulation, but less than 30, more preferably less than 25, most preferably less than 20% by weight.

Preferably, the surfactant systems of the invention have a viscosity of greater than 10 Pa*s at 2% total surfactant concentration, more preferably a viscosity of greater than 100 Pa*s at 5% total surfactant concentration, still more preferably a viscosity of greater than 1000 Pa*s at 10% total surfactant concentration, when the viscosity is measured at a shear rate of 1×10⁻³ s⁻¹ using a rheometer with a cross-hatched 40 mm diameter parallel plate and a gap of 0.8 mm.

Typically, surfactant systems comprise surfactants having from 1 to 30 carbon atoms with “classical” carbon chain lengths ranging from 19 to 30 carbon atoms providing the highest viscosity systems, as shown in, for example, in U.S. Pat. No. 6,258,859 to Dahayanake et al. Surprisingly and unexpectedly, the compositions of the invention provide high viscosities (such as those disclosed in the preceding paragraph) and/or high yield values with surfactants having shorter carbon chain lengths ranging from 8 to 18, preferably from 10 to 16, more preferably from 12 to 14 carbon atoms. The surfactant systems of the invention also surprisingly and unexpectedly achieve these high viscosities and/or yield values often using less total surfactant concentration than in classical systems. As such, the compositions of the invention unexpectedly provide more efficient and cost effective compositions capable of suspending solid and liquid particles, preferably without sedimentation.

In some embodiments, particles are suspended after 6 weeks at room temperature without heavy particles settling to the bottom or lighter particles floating to the top of the gel.

In some embodiments, optimum surfactant levels may be determined by measuring the yield point with a rheometer and selecting a concentration which provides an optimum combination of low viscosity and high yield point. A quick indication of the formation of a structured system is to shake air into the composition and observe the bubbles, which show no tendency to rise in a structured system. The resulting gel preferably also presents a characteristic vibration upon striking a container comprising the gel. The structure of the gel, for example, cubic phase or worm-like micellar system, may identified by small angle X-ray diffraction.

The weight ratio of anionic surfactant to amphoteric and/or zwitterionic surfactant is preferably greater than 1:10, more preferably greater than 1:5, even more preferably greater than 1:2, most preferably greater than 1:1.5, but less than 10:1, more preferably less than 5:1, even more preferably less than 2:1, most preferably less than 1.5:1.

The composition normally contains a non-surfactant electrolyte as a structurant. The amphoteric/zwitterionic component, when prepared from chloracetic acid, usually contains sufficient sodium chloride to supply the structurant requirement of the formulation. Additional electrolyte can usually be tolerated and may be required if a low salt amphoteric is used. The electrolyte may for example comprise sodium, potassium and/or ammonium chloride, sodium or potassium carbonate, sodium phosphate, or sodium citrate.

For many shampoo applications high levels of electrolyte are undesirable. They may be unacceptably harsh on the hair. The compositions of the invention are therefore preferably substantially free from electrolyte in excess of that required for structuring. Generally the lower the electrolyte content, the better the foaming properties. I prefer that the total concentration of electrolyte is less than 10% by weight of the composition, more preferably less than 7%, most preferably less than 5%. The minimum amount will depend on the particular surfactant mixture and the total surfactant concentration. It may readily be determined for a given formulation, in cases where the surfactant supplies insufficient electrolyte, by making incremental additions until the formulation suspends air on shaking. The structurant may comprise sugar.

A shampoo of the invention may contain cationic polymers, such as, for example a polyquaternium polymer, present in an amount effective for hair treatment. Typically this requires at least 0.01%, preferably at least 0.05%, more preferably at least 0.1%, most preferably at least 0.2% by weight of the polymer. While it is technically possible to suspend much larger concentrations of polymer, e.g. more than 10%, for reasons of cost effectiveness we prefer to use less than 5%, more preferably less than 2%, still more preferably less than 1%, most preferably less than 0.5% by weight of the polymer.

Shampoos of the invention preferably contains at least an effective amount, preferably more than 0.05, more preferably more than 0.1, most preferably more than 0.5%, by weight, based on the total weight of the composition, of an anti dandruff agent, such as selenium sulphide. The upper limit, technically, is largely dependant upon what viscosity can be tolerated.

The particle size of the selenium sulphide is preferably greater than 0.1 μm, more preferably greater than 1 μm, more preferably still, greater than 10 μm. The particles are preferably less than 1 mm, more preferably less than 0.75 mm, still more preferably less than 0.5 mm, most preferably less than 0.1 mm.

The composition may comprise dyes, suspended pigments and/or opacifiers such as mica. We particularly prefer the use of coated mica, and especially mica coated with metal oxides such as iron oxide, titanium oxide, stannic oxide aluminium oxide and or silica. Such products are available in a number of different shades, some of which are especially suitable for hair shampoos. The mica preferably has a particle size greater than 1 μm, more preferably greater than 5 μm, most preferably greater than 10 μm, but less than 200 μm, more preferably less than 100 μm, most preferably less than 50 μm.

The composition preferably contains effective amounts of hair conditioners such as oleyl alcohol, ethyl oleate, oleyl ethoxylate or glycerol, e.g. in proportions greater than 0.05, preferably greater than 0.1, more preferably greater than 0.5, most preferably greater than 0.7% by weight of the composition, but less than 10, preferably less than 5, more preferably less than 3, most preferably less than 2%.

The product preferably contains a buffer, such as a citrate/citric acid buffer. The pH is preferably greater than 4, more preferably greater than 5. Where the ingredients are sufficiently stable, the pH is still more preferably greater than 6, most preferably greater than 6.5, but less than 9, more preferably less than 8, most preferably less than 7.5. In the case of selenium sulphide the pH needs to be lower to ensure stability, e.g. below 6.

The product may optionally contain other common ingredients of shampoos, such as essential oils, mineral oils, vegetable oils, silicones, fragrances, antiseptics and topical medicaments.

The invention is useful for shampoos, shaving gels, bath gels, shower gels, cosmetic creams, ointments and other topical medicaments, exfoliants and toilet rim blocks.

The invention will be illustrated by the following examples, in which all proportions are % by weight unless stated to the contrary.

### Examples 1-13

Each of the compositions listed in the following table was prepared by stirring the commercial surfactants, together, where appropriate, with the required amount of additional water. The salt content was provided adventitiously by the commercial surfactant. In the case of examples 2, 3, 4, 5 and 13 all the active components were available as a mobile phase of sufficient concentration to provide the final composition and were simply prepared by cold mixing. In the remaining examples, one or more of the components was added as a solid powder or flake. In these examples the mixture was heated overnight at 50° C. to ensure homogeneity. In each case the product was a clear, optically isotropic gel with suspending properties. The compositions exhibited “ringing” behaviour characteristic of cubic symmetry or viscoelastic worm-like micellar systems. The compositions were able to tolerate addition of up to 5% sodium chloride without showing signs of instability, or loss of suspending power.

| Example | 1 | 2 | 3 | 4 | 5 | 6 | 7 | 8 | 9 | 10 | 11 | 12 | 13 |
|---|---|---|---|---|---|---|---|---|---|---|---|---|---|
| Na lauryl sulfate | 6.7 | 7.3 | 7.3 | | | | | | | | 5.8 | 5.8 | 3.7 |
| Na methyl oleoyl | 7.5 | | | | | | | 14.0 | | 8.7 | 5.8 | 5.8 | |
| taurate | | | | | | | | | | | | | |
| Cocamidopropyl | | 14.7 | | 14.7 | | 16.5 | | | 11.6 | 8.7 | 5.8 | | 7.3 |
| hydroxy sultaine | | | | | | | | | | | | | |
| Lauramidopropyl | | | 14.6 | | 14.7 | | 16.5 | | | | | | 7.3 |
| betaine | | | | | | | | | | | | | |
| Na alpha olefin | | | | 7.3 | 7.7 | | | | | | | | 3.8 |
| sulphonate | | | | | | | | | | | | | |
| Coco fatty acid | | | | | | 5.4 | | | | | | | |
| ester of Na | | | | | | | | | | | | | |
| isethionate | | | | | | | | | | | | | |
| Coco fatty acid | | | | | | 0.6 | | | | | | | |
| Stearic acid | | | | | | 1.6 | | | | | | | |
| Na oleate | | | | | | | 7.9 | | | | | | |
| Na lauryl | | | | | | | | 3.4 | 5.8 | | | | |
| sulphoacetate | | | | | | | | | | | | | |
| Cocamidopropyl | | | | | | | | | | | | 5.8 | |
| betaine | | | | | | | | | | | | | |
| Sodium Chloride | 4.2 | 2.3 | 2.4 | 2.3 | 2.4 | 2.6 | 2.7 | 4.75 | 3.0 | 5.8 | 2.6 | 2.4 | 2.4 |
| Sodium Sulfate | 0.75 | 0.1 | 0.1 | 0.1 | 0.1 | | | 0.5 | 0.9 | | 0.1 | 0.1 | 0.1 |
| Water | 80.85 | 75.6 | 75.6 | 75.6 | 75.1 | 73.3 | 72.9 | 77.35 | 78.7 | 76.8 | 79.9 | 80.1 | 75.4 |

### Examples 14 and 15

The following examples were prepared by cold mixing by hand with spatula at room temp.

| Example | 14 | 15 |
|---|---|---|
| Sodium Lauryl Sulfate | 1.7 | 6.5 |
| Cocamido propylhydroxy sultaine | 4.3 | 13.0 |
| Sodium Chloride | 0.7 | 2.0 |
| Mica - ‘Candurin Gold Sheen’ | 0.2 | |
| Soybean Oil | | 1.9 |
| Water | 93.1 | 76.6 |

Example 14 was thin and non-ringing.

Example 15 was a milky ringing gel.

Both samples were supporting mica and oil after 6 weeks at room temp without the mica sedimenting or the oil floating to the surface.

### Example 16

### Phase Diagram Sulfate/Betaine 0% NaCl

Sodium alkyl sulfate in the form of Rhodapon LCP (29% w/w active, 0.05% NaCl) was mixed with lauramidopropyl betaine in the form of Mackam DAB-ULS (35% w/w active, 0.34% NaCl). 100 g samples were cold mixed by hand, stored overnight at 60° C., and allowed to cool to room temperature. The following results were obtained:

| w/w ratio of | | 10% | 15% | 20% | 25% | 30% | |
|---|---|---|---|---|---|---|---|
| Sulfate:Betaine | 5% actives | actives | actives | actives | actives | actives | 35% actives |
| 2:1 | | L1 | L1 | L1 | L1/GEL | L1/GEL | |
| 1:1 | | | | | | | |
| 1:2 | | L1 | L1 | L1/M | L1/M | M | |
| 1:3 | | | | | | | |
| 1:4 | | L1 | L1 | L1 | L1/M | M | |

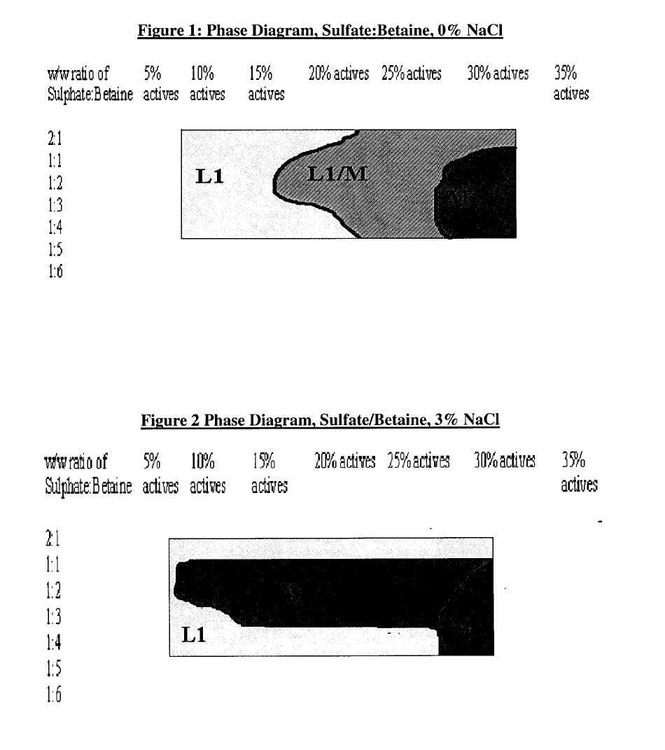

These results were used to construct the phase diagram shown in FIG. 1, which shows a “conventional” L1-M transition with increasing surfactant. No ringing gels were observed.

### Example 17

### Phase Diagram, Sulfate/Betaine, 3% NaCl

The feedstocks and method were the same as Example 16. Samples were doped with sodium chloride and the following results were obtained:

| w/w ratio of | | 10% | 15% | 20% | 25% | 30% | |
|---|---|---|---|---|---|---|---|
| Sulfate:Betaine | 5% actives | actives | actives | actives | actives | actives | 35% actives |
| 2:1 | | L1 | L1 | L1 | L1 | L1 | |
| 1:1 | | | | | | | |
| 1:2 | | RG | RG | RG | RG | RG/M | |
| 1:3 | | L1 | RG | RG | RG | M | |
| 1:4 | | L1 | L1 | L1 | L1 | M | |

These results were used to construct the phase diagram shown in FIG. 2, which shows a ringing gel phase, RG, topped and tailed by an L1 phase and an M phase at high surfactant concentrations. For 1:1 to 1:3 w/w ratios of Sulfate:Betaine, ringing gels were observed at around 0.5% salt up to around 8.0% salt. Comparing Examples 16 and 17, it was observed that ringing gel formation occurred only in the presence of a salt.

### Example 18

### Varying the Salt Concentration

From a 1:2 Sulfate:Betaine w/w system with 20% total surfactant, a set of samples from 0% to 12% sodium chloride was prepared. The following results were obtained with the salt present in the feedstocks included:

| Salt Level w/w | Result |
|---|---|
| 0.4 | weak transparent non-ringing gel |
| 0.65 to 6.15 | transparent ringing gels |
| 9.15 to 12.15 | Thick pourable clear homogenous liquids |

### Example 19

### Ringing Gel Melting Points

The melting points of the ringing gels of Example 18 were determined by inserting a thermometer through the jar cap, heating the samples to 60° C., and then cooling the samples in an ice water bath with intermittent tapping until the gel could be felt ‘ringing.’

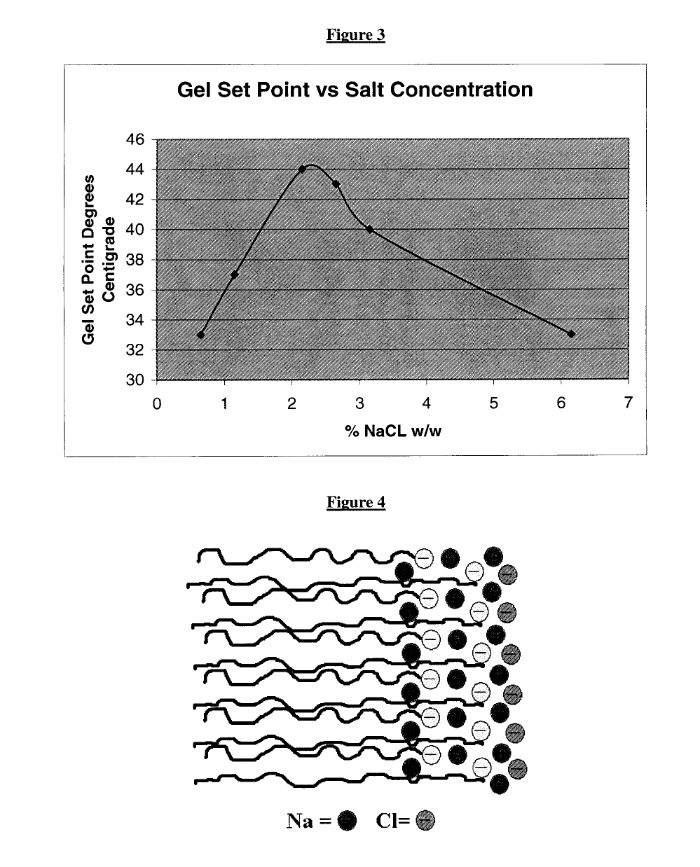

The melting points of the gels were found to vary significantly with salt concentration as shown in FIG. 3. The strongest (highest melting) gel was obtained at about 2.5% NaCl. This gel was rigid and transparent, yet flowed readily.

Unexpectedly, the strongest gel was obtained with equimolar amounts of salt and surfactant, which is consistent with, for example, the molecular packing structure shown in FIG. 4. Moreover, the amount of salt required to give the highest melting gel may be predicted by the molecular weight of the component surfactants.

### Example 20

### SLE(1)S+Betaine

Feedstock:

Mirataine BB (30% actives, 5% NaCl)
Rhodapex ESY STD (25% actives, 0.5% NaCl)

Samples were prepared at 15%, 20%, and 25% total surfactant at 1:2 w/w SLE(1)S:BB and 1:3 w/w SLE(1)S:BB. Samples were mixed by hand at room temperature.

It was found that all samples were a mixture of dispersed clear gel droplets in thin L1 phase. The samples were stored overnight at 60° C. and then re-examined.

At room temperature, all the 1:2 w/w samples were clear isotropic ringing gels, and the 1:3 samples comprised lumps of clear gel in thick L1.

### Example 21

### Alternative Electrolytes

Using the feedstocks of Example 20, samples were prepared at 1:2 w/w Alkyl Sulfate:Betaine with 20% total surfactant. Samples with the following molar equivalent amounts of electrolyte were prepared:
(a) 2.4% sodium chloride
(b) 3.16% sodium carbonate
(c) 5.0% trisodium citrate
(d) 5.3% potassium tripolyphosphate
(e) 4.3% calcium chloride

Strong ringing gels were obtained for samples (a) to (d). No physical differences were observed between the gels except for sample (d), which was cloudy. Sample (e) was biphasic with a creamy yellow precipitate of Ca(AS)₂.

### Example 22

### Phase Diagram for SLE(2)S:Betaine System

Feedstock:

Sodium lauryl ether (2 mole) sulfate (“SLE(2)S’) from Surfac LC/X 27.5% w/w actives Lauramidopropyl betaine (sodium salt) from Mirataine BB 30% w/w actives, 5% NaCl

The following samples were cold mixed, then stored at 60° C. overnight before cooling to room temperature:

| w/w ratio of | | 10% | 15% | 20% | 25% | 30% | |
|---|---|---|---|---|---|---|---|
| SLE(2)S:Betaine | 5% actives | actives | actives | actives | actives | actives | 35% actives |
| 2:1 | | | L1 | G | RG | | |
| 1:1 | | | G | RG | RG | | |
| 1:2 | | L1 | L1 | G | RG | M | |
| 1:3 | | | L1 | L1/G | L1/G | | |

L1 = clear micellar
G = clear gel, non ringing
RG = clear ringing gel
L1/G = clear gel in L1
M = thick cloudy gel

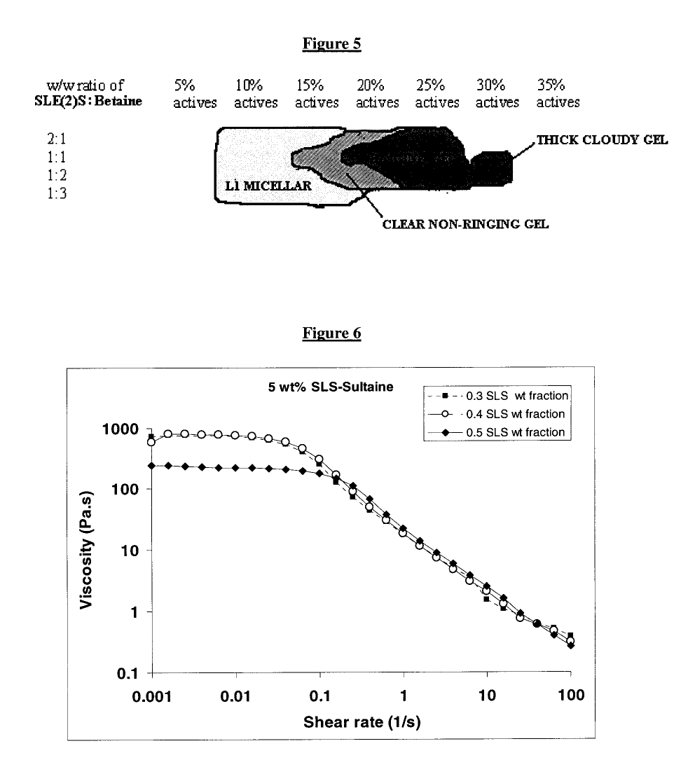

A comparatively smaller RG phase with a less rigid gel was observed as shown in FIG. 5. The strongest ringing gels were observed at 1:1 w/w ratio (about 1:1 moles).

### Example 23

### SLS and Sultaine System

All formulations prepared with products that were obtained from Rhodia Inc. Surfactants used were Mackam CBS 50G (cocamidopropylhydroxysultaine) (“Sultaine”) and SLS (sodium lauryl sulfate) along with deionized water. Sample formulations were prepared with a constant salt concentration of 2.14 wt %. For this purpose the following stock solutions were prepared (i) Sultaine, Salt and Water (ii) SLS and Salt and (iii) Salt and Water. Charges from each of the stock solutions were added to prepare formulations at 5% and 10% by wt. actives so that the each formulation weighed 100 grams. These solutions were added in a 4 oz. glass bottle followed by cold mixing using an overhead mixer. Each solution was agitated for 3 minutes at 11-14 rpm, followed by storage in an oven at 45° C. for 3.5 hours. Following heat treatment, some solutions were centrifuged (SMI, Model—IEC HN SII) at 300 rpm for 15 minutes to remove further air bubbles. An amount of 0.1 wt % of a Glydant solution (55 wt % actives; Lonza Group) was also added to act as a preservative. The pH of all formulations was adjusted by treatment with 0.02-0.05 grams of lab prepared 25% citric acid.

Rheological measurements were carried out in an AR-G2 rheometer (TA Instruments) fitted with a Peltier Plate heating stage. A tool comprising a Plane SST ST with cross-hatched 40 mm diameter parallel plate geometry was used. A solvent cap was used to prevent loss due to evaporation and prevent change in sample composition. Small amounts of samples were scooped out using pipettes, and, after adjusting the geometric gap corresponding to the aforementioned tool, the excess sample accumulating at the rim of the tool was scooped out.

Two types of rheology studies were performed: steady state studies and oscillatory shear studies. In steady state rheological measurements, the affect of viscosity and shear stress as a function of shear rate was measured. The shear rate was varied from 0.001 (1/s) to 10 (1/s) and the temperature was held constant at 25° C. In oscillatory shear measurement studies, a small angle sinusoidal deformation was applied to the sample so as not to disturb fluid structure. The rheometer was operated in dynamic mode and the viscoelastic modulii (G′ and G″) were measured as a function of oscillation frequency. The range of oscillation frequency was varied from 0.01 rad/s to 100 rad/s. All samples underwent a 3.0s pre shear at 1 Pa, followed by a 3.0s equilibration prior to viscosity, shear stress, and viscoelastic modulii measurements.

FIG. 6 shows the steady shear rate viscosity curve for three compositions of sodium lauryl sulfate and cocoamidopropylhydroxysultaine at 5 wt % of total surfactant concentration and 2.14 wt % NaCl at 0.3 SLS wt fraction (black squares), 0.4 SLS wt fraction (open circles), and 0.5 SLS wt fraction (black diamonds).

FIG. 7 shows the dynamic shear modulii for a sodium lauryl sulfate and cocoamidopropylhydroxysultaine mixture at 5 wt % of total surfactant concentration and 2.14 wt % NaCl at 0.4 SLS wt fraction.

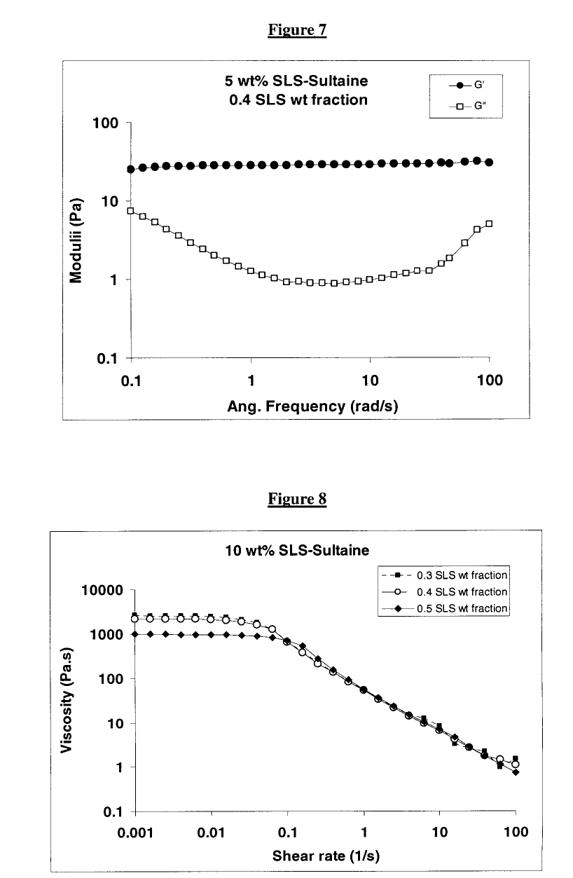

FIG. 8 shows the steady shear rate viscosity curve for three compositions of sodium lauryl sulfate and cocoamidopropylhydroxysultaine at 10 wt % of total surfactant concentration and 2.14 wt % NaCl at 0.3 SLS wt fraction (black squares), 0.4 SLS wt fraction (open circles), and 0.5 SLS wt fraction (black diamonds).

What is claimed is:

1. A composition comprising a mixture of at least one anionic surfactant and at least one zwitterionic or amphoteric surfactant, wherein:
said at least one anionic surfactant comprises a C₈-C₁₈ carbon chain;
said at least one zwitterionic or amphoteric surfactant comprises a C₈-C₁₈ carbon chain; and
said composition comprises a Pourable ringing gel capable of suspending solid or liquid particles without sedimentation.

2. The composition of claim 1, wherein at least one of the carbon chains is at least partially unsaturated.

3. The composition of claim 1, wherein at least one of the carbon chains is branched.

4. The composition of claim 1, wherein the surfactants do not comprise carbon chains having more than 18 carbon atoms.

5. The composition of claim 1, wherein the surfactants do not comprise carbon chains having more than 16 carbon atoms.

6. The composition of claim 1, wherein the surfactants do not comprise carbon chains having more than 14 carbon atoms.

7. The composition of claim 1, wherein the at least one anionic surfactant comprises a C₁₀-C₁₆ carbon chain.

8. The composition of claim 7, wherein the at least one anionic surfactant comprises a C₁₂-C₁₄ carbon chain.

9. The composition of claim 1, wherein the at least one anionic surfactant comprises a C₁₂₋₁₄ alkyl sulfate, an oleyl taurate, or a mixture thereof.

10. The composition of claim 1, wherein the anionic surfactant comprises a dodecanoate, myristate, stearate, isostearate, oleate, linoleate, linolenate, behenate, erucate, palmitate, coconut or tallow soap; olefin sulphonate; paraffin sulphonate; isethionate; sarcosinate; sulphosuccinate; sulphosuccinamate or a combination thereof.

11. The composition of claim 1, wherein the anionic surfactant is not an ethoxylated surfactant.

12. The composition of claim 1, wherein the at least one at least one zwitterionic or amphoteric surfactant comprises a C₁₀-C₁₆ carbon chain.

13. The composition of claim 12, wherein the at least one zwitterionic or amphoteric surfactant comprises a C₁₂-C₁₄ carbon chain.

14. The composition of claim 1, wherein the at least one zwitterionic or amphoteric surfactant comprises an imidazoline betaine.

15. The composition of claim 1, wherein the at least one zwitterionic or amphoteric surfactant comprises a compound of formula: 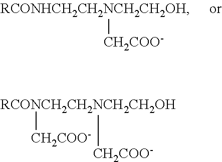
wherein R comprises said C₈-C₁₈ carbon chain.

16. The composition of claim 15, wherein said carbon chain comprises a mixture of alkyl and alkenyl groups.

17. The composition of claim 1, wherein the zwitterionic surfactant comprises a betaine, sulfobetaine or hydroxy sulfobetaine.

18. The composition of claim 17, wherein the zwitterionic surfactant comprises a compound of formula:
R″R′₂NCH₂XOH
wherein:
X comprises C═O or SO₂,
R′ comprises an aliphatic group having 1 to 4 carbon atoms, and
R″ comprises an aliphatic group having from 8 to 18 carbon atoms.

19. The composition of claim 18, wherein R″ is branched.

20. The composition of claim 18, wherein R″ comprises a straight chain alkyl or alkenyl group.

21. The composition of claim 18, wherein R″ comprises a group of formula:
RCONR′(CH₂)ₙ
wherein:
R comprises said C₈-C₁₈ carbon chain
R′ comprises an aliphatic group having 1 to 4 carbon atoms, and
n is an integer ranging from 2 to 4.

22. The composition of claim 1, wherein the zwitterionic surfactant comprises a compound of formula:
R″R′₂NCH₂XOH
wherein:
X comprises C═O or SO₂,
R′ comprises a methyl, carboxymethyl, ethyl, hydroxyethyl, carboxyethyl, propyl, isopropyl, hydroxypropyl, carboxypropyl, butyl, isobutyl or hydroxybutyl group, and
R″ comprises an aliphatic group having from 8 to 18 carbon atoms.

23. The composition of claim 1, wherein the mixture further comprises a nonionic surfactant comprising an amine oxide, a polyglyceryl fatty ester, a fatty acid ethoxylate, a fatty acid monoalkanolamide, a fatty acid dialkanolamide, a fatty acid alkanolamide ethoxylate, a propylene glycol monoester, a fatty alcohol propoxylate, an alcohol ethoxylate, an alkyl phenol ethoxylate, a fatty amine alkoxylate, a fatty acid glyceryl ester ethoxylate, a mixed ethylene oxide/propylene oxide block copolymer, an ethylene glycol monoester, an alkyl polyglycoside, an alkyl sugar ester, an ethoxylated sorbitan ester, an alkyl capped polyvinyl alcohol, an alkyl capped polyvinyl pyrrolidone, or a combination thereof.

24. The composition of claim 1, further comprising at least one electrolyte.

25. The composition of claim 24, wherein the electrolyte comprises sodium, potassium and/or ammonium chloride; sodium or potassium carbonate; sodium phosphate;
sodium citrate, sugar, or a combination thereof.

26. The composition of claim 25, wherein the electrolyte comprises sodium chloride.

27. The composition of claim 24, wherein the total amount of electrolyte is less than 10% by weight of the composition.

28. The composition of claim 27, wherein the total amount of electrolyte is less than 5% by weight of the composition.

29. The composition of claim 1, wherein the gel comprises about 20% of total surfactant comprising equimolar amounts of anionic and amphoteric surfactants, and sufficient electrolyte to balance charges on the surfactants.

30. The composition of claim 1, wherein the total amount of surfactant comprises greater than 2% of the composition by weight.

31. The composition of claim 30, wherein the total amount of surfactant comprises greater than 4% of the composition by weight.

32. The composition of claim 31, wherein the total amount of surfactant comprises greater than 6% of the composition by weight.

33. The composition of claim 32, wherein the total amount of surfactant comprises greater than 8% of the composition by weight.

34. The composition of claim 33, wherein the total amount of surfactant comprises greater than 15% of the composition by weight.

35. The composition of claim 1, wherein the total amount of surfactant ranges from 8% to 30% of the composition by weight.

36. The composition of claim 1, wherein the weight ratio of the at least one anionic surfactant to the at least one amphoteric or zwitterionic surfactant ranges from 1:10 to 10:1.

37. The composition of claim 1, wherein the weight ratio of the at least one anionic surfactant to the at least one amphoteric or zwitterionic surfactant ranges from 1:5 to 5:1

38. The composition of claim 1, wherein the weight ratio of the at least one anionic surfactant to the at least one amphoteric or zwitterionic surfactant ranges from 1:2 to 2:1

39. The composition of claim 1, wherein the weight ratio of the at least one anionic surfactant to the at least one amphoteric or zwitterionic surfactant ranges from 1:1.5 to 1.5:1

40. The composition of claim 1, wherein said composition is a component in a personal care product.

41. The composition of claim 1, wherein said composition comprises a personal care product.

42. The composition of claim 41, wherein said personal care product comprises a shampoo, shaving gel, bath gel, shower gel, cosmetic cream, ointment, or exfoliant.

43. The composition of claim 1, wherein said surfactants form a worm-like micellar structure.

44. The composition of claim 1, wherein said surfactants form a structure comprising a cubic symmetry.

45. The composition of claim 1, wherein the composition is clear and optically isotropic.

46. The composition of claim 1, wherein the composition is shear thinning.

47. The composition of claim 1, further comprising suspended solid or liquid particles.

48. The composition of claim 47, wherein the solid or liquid particles comprise an oil, an anti-dandruff agent, a hair conditioner, an opacifier, mica, or a metallic flake.

49. The composition of claim 47, wherein the hair conditioner comprises a cationic polymer.

50. The composition of claim 48, wherein said particles comprise an anti-dandruff agent.

51. The composition of claim 50, wherein said anti-dandruff agent comprises selenium sulfide.

52. The composition of claim 48, wherein the particles comprise mica.

53. The composition of claim 1, wherein said composition has a viscosity greater than Pa*s at 2% total surfactant concentration when the viscosity is measured at a shear rate of 1×10⁻³ s⁻¹ with a rheometer comprising a cross-hatched 40 mm diameter parallel plate and a gap of 0.8 mm.

54. The composition of claim 53, wherein the viscosity is greater than 100 Pa*s.

55. The composition of claim 54, wherein the viscosity is greater than 1000 Pa*s.

56. The composition of claim 1, wherein:
said at least one anionic surfactant comprises a C₁₀-C₁₆ carbon chain, and/or
said at least one zwitterionic or amphoteric surfactant comprises a C₁₀-C₁₆ carbon chain; and
further wherein said composition has a viscosity greater than 10 Pa*s at 2% total surfactant concentration when the viscosity is measured at a shear rate of 1×10⁻³ s⁻¹ with a rheometer comprising a cross-hatched 40 mm diameter parallel plate and a gap of 0.8 mm.

57. The composition of claim 56, wherein the viscosity is greater than 100 Pa*s.

58. The composition of claim 57, wherein the viscosity is greater than 1000 Pa*s.

59. The composition of claim 1, wherein:
said at least one anionic surfactant comprises a C₁₂-C₁₄ carbon chain, and/or
said at least one zwitterionic or amphoteric surfactant comprises a C₁₂-C₁₄ carbon chain; and
further wherein said composition has a viscosity greater than 10 Pa*s at 2% total surfactant concentration when the viscosity is measured at a shear rate of 1×10⁻³ s⁻¹ with a rheometer comprising a cross-hatched 40 mm diameter parallel plate and a gap of 0.8 mm.

60. The composition of claim 59, wherein the viscosity is greater than 100 Pa*s.

61. The composition of claim 60, wherein the viscosity is greater than 1000 Pa*s.

62. The composition of claim 1, wherein the composition has a pH ranging from 4 to 9.

63. The composition of claim 62, wherein the composition has a pH ranging from 6 to 8.

64. A composition comprising a mixture of sodium lauryl sulfate, cocoamidopropylhydroxysultaine, and sodium chloride in an aqueous medium,
wherein said composition comprises a Pourable ringing gel capable of suspending solid or liquid particles, and
further wherein said composition has a viscosity greater than 10 Pa*s at 2% total surfactant concentration when the viscosity is measured at a shear rate of 1×10⁻³ s⁻¹ with a rheometer comprising a cross-hatched 40 mm diameter parallel plate and a gap of 0.8 mm.

65. A method of making a surfactant composition comprising:
mixing at least one anionic surfactant and at least one zwitterionic or amphoteric surfactant in an aqueous medium, wherein:
said at least one anionic surfactant comprises a C₈-C₁₈ carbon chain;
said at least one zwitterionic or amphoteric surfactant comprises a C₈-C₁₈ carbon chain; and
said composition comprises a Pourable ringing gel capable of suspending solid or liquid particles without sedimentation.

66. A method for suspending solid or liquid particles comprising mixing said particles with a composition comprising a mixture of at least one anionic surfactant and at least one zwitterionic or amphoteric surfactant, wherein:
said at least one anionic surfactant comprises a C₈-C₁₈ carbon chain;
said at least one zwitterionic or amphoteric surfactant comprises a C₈-C₁₈ carbon chain; and
said composition comprises a Pourable ringing gel capable of suspending said particles without sedimentation.
